SEQUENCE LISTING (26 sequences)

SEQ ID NO: 1 - DNA, 205 bp, Avian leukosis/sarcoma virus of the family Retroviridae
  Subgroup A of the avian leukosis/sarcoma virus.
ctacagctgttaggttcccagtctctccctaacattactaatattactcagatctccggtgtaaccgggggatgcgtaggcttcaggccaaaaggggttccttggtatctgggttggtctagacaggaagccacgcggtttctccttagacgcccctctttctctaactcctcgaaaccgtttacagtggtgacagcggataggc

SEQ ID NO: 2 - DNA, 229 bp, Avian leukosis/sarcoma virus of the family Retroviridae
  Subgroup B of the avian leukosis/sarcoma virus.
ctacaactgctaggttcccagtctctccccaatataactaatattactcggatccccagtgtggctggaggatgcataggctttaccccatacgatagtccggctggtgtctacggatgggaccggagagaggttacacacatccttctgaccgacccagggaacaatcctttctttgataaggcctctaactcctcgaaaccgtttacagtagtgacagcggacaggc

SEQ ID NO: 3 - DNA, 211 bp, Avian leukosis/sarcoma virus of the family Retroviridae
  Subgroup C of the avian leukosis/sarcoma virus.
ctgcagctgctaggttcccagtctctccctaacgttactaacattactcaggtctctggcgtggccgggggatgtgtatatttcgccccaagggccactggcctgtttttaggttggtctaaacaaggtctctcgcggttcctcctccgtcacccctttacctccacctctaactccacggaaccgttcacggtggtgacagcggatagac

SEQ ID NO: 4 - DNA, 229 bp, Avian leukosis/sarcoma virus of the family Retroviridae
  Subgroup D of the avian leukosis/sarcoma virus.
ctgcagctgttaggctcccagtctctccctaatatcgctaatattactcagatccctggtgtggcaggaggatgcataggcttcaccccatacggcagtccggctggtgtttacgggtggggccgggaagaggtgacacacatcctcttaaccaacccccctgataatcctttctttaaccgtgcttctaactccacggaaccgtttacggtggtgacagcggataggc

SEQ ID NO: 5 - DNA, 228 bp, Avian leukosis/sarcoma virus of the family Retroviridae
  Subgroup E of the avian leukosis/sarcoma virus.
ctacagctgctaggttcccagtctctccctaacattactaatattactcagatttctggtgtaaccgggggatgcgtaggcttcgccccacactccaatccaagtggtgtctacgggtggggccggagacaggttacacacaacttcttgatcgccccgtgggtcaatcctttctttaacagcgcttctaactccacggaaccgttacggtggtgacagcggataggc

SEQ ID NO: 6 - DNA, 202 bp, Single Comb White Leghorn chicken
  PCR product Fb2, or 2F, isolated from egg albumin of ALSV-positive stock F chicken
acagctgttaggttcccagtttttcctcacattattaatattactcaaatttctggtgtaaccggaggaggcgtaggctttagaccaggagggatcccctggtatataggatggactagacaggaagccacacggttcctccttagacaatcctccttttctaattccacggaaccatttacggtggtgacagcggataggc

SEQ ID NO: 7 - DNA, 22 bp, artificial sequence
  primer_bind: Forward primer PA1 specific for the detection of viral subgroup A of avian/leukosis sarcoma virus.
ctacagctgttaggttcccagt

SEQ ID NO: 8 - DNA, 21 bp, artificial sequence
  primer_bind: Reverse primer PA2 specific for the detection of viral subgroup A of avian/leukosis sarcoma virus.
gcctatccgctgtcaccactg

SEQ ID NO: 9 - DNA, 202 bp, Single Comb White Leghorn chicken
  RT-PCR product from egg albumin of stock 2F chicken.
acagctgttaggttcccagtttttccctcacattataatattactcaaatttctggtgtaaccggaggaggcgtaggctttagaccaggagggatcccctggtatataggatggactagacaggaagccacacggttcctccttagacaatcctccttttctaattccacggaaccatttacggtggtgacagcggataggc

SEQ ID NO: 10 - DNA, 226 bp, Single Comb White Leghorn chicken
  RT-PCR product from egg albumin of stock 6F chicken.
cagctgttaggttcccagtctctccctaacattactaatattactcagatttctggtgtaactgggggatgcgtaggcttcaccccacactccaatccaagtggtgtttacgggtggggccggagacaggttacacacaacctcttgatcgccccgtgggtcaatcctttctttaacagcgcttctaactccacggaaccgtttacggtggtgacagcggataggc

SEQ ID NO: 11 - DNA, 225 bp, Single Comb White Leghorn chicken
  RT-PCR product from egg albumin of stock 7Q chicken.
cagctgttaggttcccagtttctccctaacattattaatattactcagatttctggtgtaactgggggatgcgtaggcttcaccccacactccaatccaagtggtgtttacgggtggggccggagacaggttacacacaacttcttgatcgccccgtgggtcaatcctttctttaacagcgcttctaactccacggaaccgtttacggtggtgacagcggatagg

SEQ ID NO: 12 - DNA, 229 bp, Single Comb White Leghorn chicken
  RT-PCR product from egg albumin of stock 10Q chicken.
ctacagctgttaggttcccagtctctccctaacattactaatattactcagatttctggtgtaaccgggggatgcgtaggcttcgccccacactccaatccaagtggtgtctacgggtggggccggagacaggttacacacaacttcttgatcgccccgtgggtcaatcctttctttaacagcgcttctaactccacggaaccgtttacggtggtgacagcggataggc

SEQ ID NO: 13 - DNA, 224 bp, unknown
  RT-PCR product from egg albumin of commercial chicken from randomly chosen grocery store # 205.
gctgttaggttcccagtctctccctaacattactaatattactcagatttctggtgtaaccgggggatgcgtaggcttcaccccacactccaatccaagtggtgtttacgggtggggccggagacaggttacacacaacttcttgatcgccccgtgggtcaatcctttctttaacagcgcttctaactccacggaaccgtttacggtggtgacagcggataggc

SEQ ID NO: 14 - DNA, 203 bp, unknown
  RT-PCR product from egg albumin of commercial chicken from randomly chosen grocery store # 65.
tacagctgttaggttcccagtctctccctaacattactaacatactcaaatttctggtgtaaccggaggatgcgtaggctttagaccaggagggatcccctggtatatgggatggactagacaggaagccacacggttcctccttaaacaatcctccttttctaattccacggaaccatttacggtggtgacagcggataggc

SEQ ID NO: 15 - DNA, 21 bp, artificial sequence
  primer_bind 5564..5585: Forward primer PU1 specific for the detection of viral subgroup A-E of avian/leukosis sarcoma virus.  Position corresponds to the numbering of the RNA genome of the Prague C strain of RSV.
ctrcarctgytaggytcccag

SEQ ID NO: 16 - DNA, 21 bp, artificial sequence
  primer_bind 5772..5791: Reverse primer PU2 specific for the detection of viral subgroup A-E of avian/leukosis sarcoma virus.  Position corresponds to the numbering of the RNA genome of the Prague C strain of RSV.
gycaycactgtcgcctrtccg

SEQ ID NO: 17 - DNA, 20 bp, artificial sequence
  primer_bind 5642..5661: Forward primer PA10 specific for the detection of viral subgroup A of avian/leukosis sarcoma virus.  Position corresponds to the numbering of the RNA genome of the Prague C strain of RSV.
ggcttcaggccaaaaggggt

SEQ ID NO: 18 - DNA, 22 bp, artificial sequence
  primer_bind 5858..5879: Reverse primer PA20 specific for the detection of viral subgroup A of avian/leukosis sarcoma virus.  Position corresponds to the numbering of the RNA genome of the Prague C strain of RSV.
gtgcattgccacagcggtactg

SEQ ID NO: 19 - DNA, 20 bp, artificial sequence
  primer_bind 5642..5661: Forward primer PB1 specific for the detection of viral subgroup A of avian/leukosis sarcoma virus.  Position corresponds to the numbering of the RNA genome of the Prague C strain of RSV.
ggctttaccccatacgatag

SEQ ID NO: 20 - DNA, 21 bp, artificial sequence
  primer_bind 5861..5882: Reverse primer PB2 specific for the detection of viral subgroup A of avian/leukosis sarcoma virus.  Position corresponds to the numbering of the RNA genome of the Prague C strain of RSV.
acacatcctgacagatggacc

SEQ ID NO: 21 - DNA, 20 bp, artificial sequence
  primer_bind 5642..5661: Forward primer PC1 specific for the detection of viral subgroup C of avian/leukosis sarcoma virus.  Position corresponds to the numbering of the RNA genome of the Prague C strain of RSV.
tatttcgccccaagggccac

SEQ ID NO: 22 - DNA, 22 bp, artificial sequence
  primer_bind 5858..5879: Reverse primer PC2 specific for the detection of viral subgroup C of avian/leukosis sarcoma virus.  Position corresponds to the numbering of the RNA genome of the Prague C strain of RSV.
ccacgtctccacagcggtaagt

SEQ ID NO: 23 - DNA, 20 bp, artificial sequence
  primer_bind 5642..5661: Forward primer PD1 specific for the detection of viral subgroup D of avian/leukosis sarcoma virus.  Position corresponds to the numbering of the RNA genome of the Prague C strain of RSV.
ggcttcaccccatacggcag

SEQ ID NO: 24 - DNA, 21 bp, artificial sequence
  primer_bind 5858..5882: Reverse primer PD2 specific for the detection of viral subgroup D of avian/leukosis sarcoma virus.  Position corresponds to the numbering of the RNA genome of the Prague C strain of RSV.
ccatacgtcctcacagataga

SEQ ID NO: 25 - DNA, 20 bp, artificial sequence
  primer_bind 5642..5661: Forward primer PE1 specific for the detection of viral subgroup E of avian/leukosis sarcoma virus.  Position corresponds to the numbering of the RNA genome of the Prague C strain of RSV.
ggcttcgccccacactccaa

SEQ ID NO: 26 - DNA, 22 bp, artificial sequence
  primer_bind 5867..5888: Reverse primer PE2 specific for the detection of viral subgroup E of avian/leukosis sarcoma virus.  Position corresponds to the numbering of the RNA genome of the Prague C strain of RSV.
gcacatctccacaggtgtaaat

(19) United States
(12) Patent Application Publication
(10) Pub. No.: US 2005/0042597 A1
(43) Pub. Date: Feb. 24, 2005

(54) Viral detection system

(76) Inventor: Thuy Diem Pham, Folsom, LA (US)

Correspondence Address: THE H.T. THAN LAW GROUP, 1010 WISCONSIN AVENUE NW SUITE 580, WASHINGTON, DC, 20007, US

(21) Appl. No.: 09/866,261

(22) Filed: May 25, 2001

Related U.S. Application Data
(62) Division of application No. 09/159,325, filed on Sep. 23, 1998, now abandoned.
(60) Provisional application No. 60/061,287, filed on Oct. 7, 1997.

(51) Int. Cl. C12Q 1/70; C12Q 1/68; C12P 19/34

(52) U.S. Cl. 435/5; 435/6; 435/91.2

(57) ABSTRACT

RT-PCR based systems for detection of exogenous and endogenous avian leukosis/sarcoma viruses in the albumen of fertilized and unfertilized poultry eggs have been developed. Such systems permit the identification of virus-contaminated eggs and of viral presence in sera of birds. These systems aid in the prevention of high loss rates of egg-laying birds in the poultry industry and the reduction in potential human exposure to these oncogenic retroviruses through consumption of contaminated commercial eggs. It is further envisioned that detection methods can be modified to include viral detection of other samples from various avian origins and other animal or human retroviruses.

### CROSS-REFERENCE TO RELATED APPLICATION

This application claims the benefit of provisional application Ser. No. 60/061,287, filed Oct. 7, 1997, now abandoned.

### BACKGROUND OF THE INVENTION

1. Field of the Invention

The present invention relates generally to the fields of poultry virology and oncogenesis and cancer epidemiology in humans. More particularly, the present invention relates to a method of detection of avian leukosis/sarcoma viruses in chicken eggs.

2. Description of the Related Art

Avian leukosis/sarcoma viruses (ALSVs) are C-type RNA tumor viruses in the family Retroviridae. These viruses can cause leukemias/lymphomas and other cancers in chickens. As C-type viruses, the ALSV replication cycle includes budding from host membranes and post-budding maturation to become fully infectious viral particles. The ALSV viral genome is composed of two identical RNA fragments, which vary in size from 4 to 9 kb depending on the viral strain. Inside the virion, the diploid RNA strands are joined together at the 5′ end. Replication-competent avian leukosis viruses (ALV) contain the gene sequence (5′) LTR-gag-pol-env-LTR (3′).

The avian retrovirus env gene encodes a polyprotein (90 kDa) that is processed into two glycoproteins: gp85^{env} which appears as knob-like structures on the surface of a retrovirus particle and gp37^{env} which becomes the highly glycosylated protein spikes that link the knobs to the viral lipid envelope (Bova, C. A., et al., J. Virol., 62:75-83 (1988)). Variations in the nucleotide sequence of the gp85^{env} define ALSV subgroups A, B, C, D, and E. These variable regions code for specific gp85^{env} protein determinants which designate the viral host range by providing the infectious viral particle the essential tools for host cellular receptor recognition, penetration of the host plasma membrane, and viral entry into the host intracellular environment (Bova, C. A., et al., Virology, 152:343-354(1986)).

The exogenous and oncogenic avian leukosis/sarcoma subgroups A, B, C, and D are horizontally and vertically transmitted in chickens. Subgroup A ALV infections are commonly seen in egg-laying hens (Spencer, J. L., Avian Pathol., 13:599-619 (1984)). Subgroup E ALV viruses are non-oncogenic endogenous viruses in chickens that are transmitted in a non-infectious form from one generation to the next in a Mendelian fashion along with the host genes. They can also be transmitted horizontally in some cases, and have been shown to be oncogenic in certain avian hosts other than chickens. Recent molecular characterization of a newly isolated leukosis virus from meat-type chickens showed that subgroup J ALSV contains gene sequences homologous to both exogenous and endogenous viral elements (e.g. Bai, J., et al., J. Gen. Virol., 76:181-187 (1995)).

Avian leukosis/sarcoma viruses infections in commercial chickens adversely affect poultry and egg production since infected chickens may exhibit reduced growth rates, decreased egg production, and produce eggs of reduced size and quality (Gavora, J. S., et al., Avian Pathol., 11:29-38 (1982)). In addition, Avian leukosis/sarcoma virus causes tumor and sporadic deaths in sexually mature chickens. Congenital transmission accounts for the spread of Avian leukosis/sarcoma virus from one generation to the next, and the virus can also spread horizontally from chick to chick.

Severe economic losses to the poultry industry can occur because the viruses routinely cause a variety of cancers in commercial chickens destined for human consumption; occasionally up to 23% of birds are lost. In severe outbreaks, mortality from cancer can be greater than 40%. In addition, infected chickens may develop debilitating conditions, e.g., diseases such as osteopetrosis gallinarum, and have to be eliminated.

Another important consideration is the avian leukosis/sarcoma virus's impact on humans. Previous studies have shown that avian leukosis/sarcoma viruses can infect and transform human cells in vitro. (e.g. Johnson, E. S. Cancer Detect. Prev., 18:9-30 (1994)). Infection and tumors have also been induced experimentally in primates. Direct evidence that the avian leukosis/sarcoma viruses cause cancer in humans has not yet been found, however, and until recently, these viruses were generally considered harmless. Notwithstanding, epidemiological data show abnormally elevated rates of cancer in workers in the meat and poultry industries, particularly those engaged in slaughtering activities. It has recently been demonstrated that antibodies against avian leukosis/sarcoma viruses viral antigens are present in the sera of poultry slaughtering/processing plant workers, as well as individuals in the general population, thus providing evidence that avian leukosis/sarcoma virus can potentially infect humans. Hence, there is a scientific basis for considering avian leukosis/sarcoma viruses a potential public health threat.

To prevent transmission avian leukosis/sarcoma viruses, the poultry industry must identify infected eggs and chickens, and remove them from breeding populations. Several methods are currently used to detect avian leukosis/sarcoma viruses, including radioimmunoassays, phenotypic mixing tests, complement fixation tests, virus neutralization tests, immunofluorescence assays (IFA), and enzyme-linked immunosorbent assay (ELISA) (e.g. Spencer, J. L., In: G. F. De Boer (Ed.), Avian Leukosis. Developments in Veterinary Virology. Martinus Nijhoff Publishing, pp. 213-240. 1987).

Traditional methods of virus isolation and immunofluorescence assays are generally not used in the poultry industry due to their prohibitively long duration (about 2 weeks) to determine viral infection. Methods that are used have significant drawbacks, i.e., they are either cumbersome and unsuitable for use when large numbers of samples are involved, or lack sensitivity, or show false positive readings stemming from failure to adequately distinguish exogenous from endogenous viral infections (e.g., ELISA assays detect the avian leukosis/sarcoma viruses group specific antigen (gsa) which is similar in exogenous and endogenous avian leukosis/sarcoma viruses). This inability to distinguish between antigens of endogenous or exogenous origin has proven to be a serious liability in the rigorous assessment of viral transmission dynamics. Hence, the eradication of avian leukosis/sarcoma virus has not been achieved in the poultry industry, due to a lack of rapid, sensitive and effective field assays for detecting viral infection.

Currently, the poultry industry uses an ELISA assay to identify avian leukosis/sarcoma virus infected chickens, as the basis for an ALSV eradication campaign. However, avian leukosis/sarcoma viruses still persist and are evolving into different viral variants (Witter, R. L., Acta Vet. Hung., 45:251-266 (1997)). Further tests are needed to specifically detect avian leukosis/sarcoma viruses at the nucleic acid level, to distinguish subgroups and newly emerging avian leukosis/sarcoma virus variants. Although an RT-PCR assay in conjunction with restriction endonuclease digestion has been described to detect ALSV contamination of vaccines (Häuptli, D., et al., J. Virol. Meth., 66:71-81(1997)), this assay does not identify ALSV subgroups at the nucleic acid level or isolate wild-type viral RNA directly from the egg albumen. Additionally, this assay is flawed in that it utilizes embryonated eggs. As discussed infra, embryonated or fertilized eggs may yield results that are either falsely positive (due to avian leukosis endogenous proviruses potentially established in parental genes found in fertilized eggs or in maternal genes found in the vitelline membrane surrounding the egg yolk) or falsely negative (due to inactivation of avian leukosis/sarcoma viruses by maternal antibodies, such as IgY, found in the egg yolk).

Thus, the prior art is deficient in the availability of rapid, accurate and sensitive tests to detect avian leukosis/sarcoma viruses and to distinguish subgroups and newly emerging avian leukosis/sarcoma viruses variants. The present invention fulfills this long-standing need and desire in the art.

### SUMMARY OF THE INVENTION

RT-PCR based detection systems for avian leukosis/sarcoma virus in unfertilized chicken eggs have been developed. In this assay, the virus can be directly isolated from the egg albumen and the viral RNA efficiently screened by RT-PCR. The amplified RT-PCR product is then directly sequenced, in order to determine avian leukosis/sarcoma virus viral subgroup specificity. Systems specifically designed for effective detection of avian leukosis/sarcoma virus in chicken eggs have been refined, modifications of such systems for use in adult birds are also available. The combined use of RT-PCR and direct sequencing of the RT-PCR product provides a new approach for identifying ALSV-infected poultry. Hence, the present invention makes available molecular-based diagnostic methods for the rapid detection of ALSV retroviruses for use by the poultry industry and public health agencies.

One object of the present invention is to provide a method for the detection of ALSV in albumen of unfertilized and fertilized chicken eggs, based on RT-PCR. It is further envisioned that detection methods can be modified to include detection of other viruses (especially oncogenic viruses) or microbial agents in eggs, cells, tissues or body fluids of poultry and other animals.

In one embodiment of the present invention, there is provided a method of detecting avian leukemia/sarcoma viruses at the nucleic acid level in a poultry sample, comprising the steps of: isolating viral RNA from said poultry sample; and performing RT-PCR. Representative poultry samples for the methods disclosed herein include fertilized and unfertilized chicken egg albumen, blood, feather pulp, cell lines and body fluids from chickens.

In another embodiment of the present invention, there is provided a method of determining ALSV subgroup specificity at the nucleic acid level, including distinguishing between exogenous and endogenous retroviruses, comprising the steps of: isolating viral RNA from a poultry sample; performing RT-PCR; and sequencing the amplified RT-PCR product.

In another embodiment of the present invention, there is provided an oligonucleotide specific for the detection of viral subgroup A of ALSV at the nucleic acid level, said oligonucleotide having a sequence at least 95% identical to a sequence selected from the group consisting of: (a) SEQ ID No: 7 and SEQ ID No: 8; (b) a nucleotide sequence encoding parts of the gp^{env} 85 protein; and (c) an oligonucleotide which hybridizes under stringent hybridization conditions to a oligonucleotide defined by (a) or (b). In another embodiment of the present invention, there is provided a method of detecting ALSVs at the nucleic acid level in a poultry sample, comprising the steps of isolating viral RNA from said poultry sample; and performing RT-PCR using an oligonucleotide of described above.

In another embodiment of the present invention, there is provided an oligonucleotide specific for the detection of viral subgroups A-E of ALSV at the nucleic acid level, said oligonucleotide having a sequence at least 95% identical to a sequence selected from the group consisting of: a) SEQ ID No: 15 and SEQ ID No: 16; b) a nucleotide sequence encoding the gp^{env} 85 protein; and c) an oligonucleotide which hybridizes tinder stringent hybridization conditions to an oligonucleotide defined by (a) or (b).

In another embodiment of the present invention, there is provided a method of detecting ALSV at the nucleic acid level in a poultry sample, comprising the steps of: isolating viral RNA from said poultry sample; and performing RT-PCR using an oligonucleotide disclosed herein.

In another embodiment of the present invention, there is provided a method of determining ALSV subgroup specificity at the nucleic acid level and distinguishing between exogenous and endogenous retroviruses, comprising the steps of: obtaining a specimen from a poultry sample; isolating viral RNA from said sample; performing RT-PCR using an oligonucleotide disclosed herein; and sequencing the amplified RT-PCR product.

In yet another embodiment of the present invention, there is provided a method of determining ALSV subgroup specificity at the nucleic acid level and distinguishing between exogenous and endogenous retroviruses, comprising the steps of: obtaining a specimen from a poultry sample; isolating viral RNA from said sample; performing RT-PCR using an oligonucleotide disclosed herein; and sequencing the amplified RT-PCR product.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 displays RT-PCR amplifications from isolated RNA samples of egg albumen using primers set PU1 and PU2 which can detect all avian leukosis/sarcoma virus viral subgroups (A, B, C, D and E). 10 μl of each reaction was electrophoresed through a 3% Nusieve gel, stained with ethidium bromide and visualized under ultraviolet light. Lane M contained the 123 bp ladder which served as the molecular size marker, Lane 2 contained the positive RNA control (Promega), Lane 3 contained the RNase-free water in place of template RNA, Lanes 4, 5, 6, and 7, contained egg albumen samples from stock F chickens, and Lane 8 contained the negative control albumen sample from N chickens. Note the approximate 246 bp bands in Lanes 4, 5, 6 and 7.

FIG. 2 is a nucleotide sequence comparison of the five avian leukosis/sarcoma virus subgroups A (SEQ ID No:1), B (SEQ ID No:2), C (SEQ ID No:3), D (SEQ ID No:4) and E (SEQ ID No:5), to the sequenced RT-PCR product, sample Fb2 (SEQ ID No:6) which came from the egg albumen of an ALSV-positive stock F chicken, using sense primer PA1 (SEQ ID No:7) and antisense primer PA2 (SEQ ID No:8) which flank the hypervariable region 1 of the ALSV gp85 env gene (Bova, C. A., et al., J. Virol., 62:75-83 (1988)). The sequence comparison begins at 5563 of Pr—C RSV and ends at 5792 of Pr—C RSV (Schwartz, D. E., et al., Cell, 32:853-869 (1983)). The numbers at the left denote the location of the first nucleotide in the sequence. The symbol (−) denotes a gap for missing nucleotide.

FIG. 3 shows the alignment of the ALSV subgroup E (RAV-O) and ALSV subgroup A (RAV-1) nucleotide sequences with those obtained from six RT-PCR products of egg albumen samples (2F (SEQ ID No:9), 6F (SEQ ID No:10), 7Q (SEQ ID No:11), 10Q (SEQ ID No:12), 207 (SEQ ID No:13), and 65 (SEQ ID No: 14)). Matching sequences are boxed with a thin line. The sequence comparison begins at nt5563 of Pr—C RSV and ends at nt5792 of Pr—C RSV. The locations of the PA1 (SEQ ID No:8) and PA2 (SEQ ID No:9) primers used in the RT-PCR reaction are marked with arrows. The gp85^{env} hypervariable region 1 (hr 1) is marked with a straight line. Numbers at the left represent the position of the first nucleotide in the sequence and numbers on the right represent the position of the last nucleotide in the sequence. Hyphens represent gaps inserted for optimum alignment.

### DETAILED DESCRIPTION OF THE INVENTION

The present invention is directed towards a method of detection of avian leukosis/sarcoma virus in poultry that provides information on the nucleic acid level to aid in identifying avian leukosis/sarcoma virus-infected fertilized and unfertilized eggs as well as chicken birds. In this assay, the virus is isolated directly from either egg albumen or blood or feather pulp of birds, and viral RNA is then efficiently screened by RT-PCR. The amplified RT-PCR product is then directly sequenced, in order to determine avian leukosis/sarcoma virus viral subgroup specificity. This combined approach using RT-PCR and direct sequencing of the RT-PCR product provides a new diagnostic test for identifying avian leukosis/sarcoma virus-infected poultry.

The present invention is directed to a method of detecting of ALSVs at the nucleic acid level in a poultry sample, comprising the steps of: isolating viral RNA from said poultry sample; and performing RT-PCR. In embodiments of the present invention, the above method of detecting virus is performed using various poultry samples, including albumen from fertilized and unfertilized eggs, blood, feather pulp, body fluids or cells from a chicken.

The present invention is further directed to a method of determining avian leukosis/sarcoma virus subgroup specificity at the nucleic acid level, including distinguishing between exogenous and endogenous retroviruses, comprising the steps of: obtaining a specimen from a poultry sample; isolating viral RNA; performing RT-PCR; and sequencing the amplified RT-PCR product. Representative poultry samples include chicken egg albumen, blood, feather pulp, body fluids or cells from an adult chicken.

The present invention is additionally directed to an oligonucleotide specific for the detection of viral subgroup A of avian leukosis/sarcoma virus at the nucleic acid level, said oligonucleotide having a sequence at least 95% identical to a sequence selected from the group consisting of: a) SEQ ID No: 7 and SEQ ID No: 8; b) a nucleotide sequence encoding the gp^{env} 85 protein; c) an oligonucleotide which hybridizes tinder stringent hybridization conditions to a oligonucleotide defined in (a) or (b).

The present invention is also directed to an oligonucleotide specific for the detection of viral subgroups A-E of avian leukosis/sarcoma virus at the nucleic acid level, said oligonucleotide having a sequence at least 95% identical to a sequence selected from the group consisting of: a) SEQ ID No: 15 and SEQ ID No: 16; b) an oligonucleotide encoding the gp^{env} 85 protein; c) an oligonucleotide which hybridizes tinder stringent hybridization conditions to an oligonucleotide defined by (a) or (b).

The present invention is further directed to a method of detecting avian leukosis/sarcoma virus at the nucleic acid level in a poultry sample, comprising the steps of: isolating viral RNA from said poultry sample; and performing RT-PCR using oligonucleotides defined herein.

The present invention is further directed to a method of determining avian leukosis/sarcoma virus subgroup specificity at the nucleic acid level, including distinguishing between exogenous and endogenous retroviruses, comprising the steps of: obtaining a specimen from a poultry sample; isolating viral RNA; performing RT-PCR using oligonucleotides defined herein; and sequencing the amplified RT-PCR product.

The following examples are given for the purpose of illustrating various embodiments of the invention and are not meant to limit the present invention in any fashion:

### EXAMPLE 1

Poultry Specimens Tested for Virus and Viral Antigen.

Three stocks of Single Comb White Leghorn chickens, referred to as Q, F and N, were used. Albumen samples from unfertilized eggs were used, as well as samples of feather pulp and blood from adult birds. Egg albumen from unfertilized eggs was used to: 1) limit false positive reactions due to avian leukosis endogenous proviruses potentially established in parental genes found in fertilized eggs, or in maternal genes found in the vitelline membrane surrounding the egg yolk; and 2) to limit false negative reactions due to inactivation of ALSV by maternal antibodies, such as IgY, found in the egg yolk (Yamamoto, T., et al., Hen Eggs. Their Basic and Applied Science, CRC Press, Boca Raton (1997); Kottaridis, S. D., et al., Avian Diseases, 11:65-68 (1967); Smith E. J. In: De Boer, G. F., ed. Avian Leukosis. Developments in Veterinary Virology, Martinus Nijhoff Publishing, Boston, Mass. (1987)).

Stock Q chickens (n=5) were reared by a commercial company which participates in a campaign to eradicate exogenous ALSV, but were kept under commercial conditions where they were naturally exposed to pathogens. Stock Q chickens had the following endogenous (ev) ALSV genes: ev1, ev3, ev4, ev5, ev6, ev7, and ev9 (Crittenden L. B., Crit. Rev. Poultry Biol., 3:73-109 (1991)). The stock F White Leghorn chickens (n=5) were from a commercial breeding company which does not have an ALSV control program. These birds were kept under commercial conditions where they were naturally exposed to pathogens. Stock N (n=5) was a laboratory flock of White Leghorns maintained in isolation at the Canadian Food Inspection Agency, Ontario, Canada. Stock N chickens are considered free of avian leukosis virus and other known poultry pathogens.

Samples of egg albumen were collected from stock Q, F and N chickens. For collection of egg albumen, 1 ml of albumen was drawn from each of 14 eggs from the stock Q chickens, 15 eggs from the stock F chickens and 5 eggs from the stock N chickens within a day of oviposition.

Samples of feather pulp, or blood were collected from stock F and N chickens. By squeezing the tips of chicken feathers, a sample of the feather pulp was inoculated into fresh culture medium.

Chicken blood was collected without anticoagulants and was allowed to clot and then the clot was washed with tissue culture medium to remove serum and 2 ml of medium was added to each blood clot prior to storage. All specimens were stored at −80° C. prior to testing. As control samples, ALSV of subgroup A (RAV-1) and subgroup B (RAV-2) were obtained from the United States Department of Agriculture, Avian Diseases and Oncology Laboratory in East Lansing, Mich. (Ishizaki, R. & Vogt, P. K., Virology, 30:375-387 (1966)).

### EXAMPLE 2

Tissue Culture Testing of Albumen, Feather Pulp, and Blood Samples

Egg albumen, feather pulp, and blood samples were tested for viral presence, via immunofluorescence (IFA) by inoculating samples onto cultures of chicken embryo fibroblasts (C/E), which were susceptible to subgroups A-D of avian leukosis/sarcoma virus but not subgroup E. Immunofluorescence analysis was performed on C/E cells inoculated with chicken egg albumen, feather pulp and blood specimens to detect the gsa of replicating ALSV. The C/E cells were recovered from storage at −196° C. An aliquot of C/E cells was tested for viabiiity by trypan blue staining and then 45,000 viable cells in 150 ml of medium were added to each well in a 96 well plate.

Four to 18 hours after plating of cells, the cultures were inoculated with 50 ml of egg albumen, feather pulp, or blood samples. Serial dilutions of RAV-1 and RAV-2 served as positive controls and uninoculated cultures were negative controls. After 6 days, medium from each of these cultures was inoculated onto a new culture of C/E that had been prepared as described above. Then, after a period of six more days, the new culture of C/E was tested for avian leukosis/sarcoma virus gs antigen by IFA.

### EXAMPLE 3

IFA and ELISA Analysis of Egg Albumen, Feather Pulp, or Blood Samples

Both immunofluorescence and ELISA were used to screen for viruses in chickens. Viral infection was detected by testing for avian leukosis/sarcoma virus group specific antigen (gsa) by ELISA or IFA (Spencer, J. L., In. G. F. De Boer (Ed.), Avian Leukosis. Developments in Veterinary Virology, Martinus Nijhoff Publishing, pp. 213-240. 1987). ELISA was performed on the egg albumen to detect avian leukosis/sarcoma virus gs antigens, while IFA analysis was performed on C/E cells inoculated with chicken egg albumen, feather pulp and blood specimens to detect the gs antigens of replicating ALSV.

In addition to the immunofluorescence analysis, ELISA was also performed on the egg albumen samples from the stock Q and F birds. The procedure for the antigen capture ELISA has been described (Spencer, J. L., In: G. F. De Boer (Ed.), Avian Leukosis. Developments in Veterinary Virology. Martinus Nijhoff Publishing, pp. 213-240. 1987). Briefly, ELISA plates were coated with rabbit anti-p27 IgG (which recognizes the gs antigen that envelops the viral RNA genome (Coffin, J. M. In: B. N. Fields, et al., (Eds.), Virology, Vol. 2, Lippincott-Raven Publishers, Philadelphia, pp. 1767-1847, 1996; Schwartz, D. E., et al., Cell, 32:853-869 (1983)) for 18 hours at 4° C., subsequent incubations at 37° C. for 30 minutes were with control and test albumen, and horseradish peroxidase conjugated to rabbit anti-p27 IgG (Life Sciences Inc., USA). The ELISA plates were washed with PBS-Tween and the detection substrate was 2,2′-Azinodi(ethylbenzthiazoline)sulfonate (ABTS) with hydrogen peroxide. A microplate reader (model 3550, BIO-RAD, Inc., USA) was used to determine absorbance readings at 405 nm.

Six days post inoculation (dpi) the C/E cells were fixed and tested by IFA, which can detect intracellular gs antigen in cell cultures infected with ALSV virus. The monolayers were fixed by adding 200 ml of 75% aqueous acetone, chilled at −20° C., to each well. Rabbit anti-p27 hyperimmune serum, followed by goat anti-rabbit IgG conjugated to fluorescein isothiocyanate (Sigma Biosciences, USA) were added to the wells. Incubation and washing followed the addition of each reagent. An inverted stage fluorescence microscope was used to read the reactions.

ELISA and immunofluorescence showed that 4 out of 5 stock Q chickens had the gs antigen in egg albumen, but the virus was not present in cultured C/E cells tested by immunofluorescence analysis. All five chickens of stock F were positive for avian leukosis/sarcoma virus in feather pulp and blood samples, based on immunofluorescence detection of gs antigen in inoculated C/E cultures. Egg albumen samples from stock F birds were positive by ELISA in three of three birds and by tissue culture/IFA in three of four chickens tested. ELISA assay did not detect avian leukosis/sarcoma virus gs antigen in five of five egg albumen samples from five stock N chickens.

### EXAMPLE 4

Purification of Viral RNA from Egg Albumen

RNA extraction and RT-PCR analyses were performed in separate laboratories. RNAgents Total RNA Isolation System (Promega, USA) was used to extract total RNA from the egg albumen. The entire RNA isolation procedure on all 20 egg albumen samples was performed on ice. Approximately 500 μl of egg albumen samples were denatured with 600 μl of denaturing solution containing guanidine thiocyanate and CSB buffer (Citrate/Sarcosine/β-Mercaptoethanol). A 2 ml Eppendorf tube containing the mixture was vortexed for 10 seconds. Next, 60 μl of 2M Sodium Acetate, pH 4.0, was added to the Eppendorf tube containing the mixture. The tube was inverted 10 times for thorough mixing of the reagents. Then, 600 μl of phenol:chloroform:isoamyl alcohol from the organic phase of the mixture was added to the Eppendorf tube containing the egg albumen sample. The tube was capped tightly and inverted 10 times and shaken vigorously for 10 additional seconds. The tube was placed on, ice for 30 minutes, and then centrifuged at 10,000×g for 20 minutes at 4° C. After centrifugation, the top aqueous phase of the mixture, which contains the viral RNA, was removed and transferred to another RNase-free Eppendorf tube. Extreme care was taking not to remove any lower phases, which may contain proteins or genomic DNA.

Using an RNase free pipette tip, an equal volume of isopropanol was added to the aqueous phase in the tube. The tube was thoroughly mixed, and the sample was placed at −20° C. overnight to precipitate the viral RNA. The next day, centrifugation of the sample was performed at 10,000×g for 10 minutes at 4° C. to pellet the RNA. After centrifugation, isopropanol was decanted from the sample leaving the pelleted RNA in the Eppendorf tube.

With an RNase free pipette tip, 1 ml of ice-cold 75% ethanol was added to the sample to wash off any contaminant from the pelleted RNA. The solution was pipetted up and down 10 times with an RNase-free Aerosol filter pipette tip (USA Scientific, USA) in order to break up the viral RNA pellet. The sample was centrifuged at 10,000×g for 10 minutes at 4° C. The RNA pellet was dried in a vacuum desiccator (Beckman, USA) for 10 minutes. The RNA sample was dissolved in 20 μl of Nuclease-Free Water (Promega, USA) and stored at −20° C. for later analyses.

### EXAMPLE 5

Oligonucleotide Primers for RT-PCR

For detection of retroviruses in the avian leukosis/sarcoma viral complex, seven sets of primers were synthesized at the Tulane Biochemical Core Facility, New Orleans, La., on an Applied Biosystems 392 DNA/RNA Synthesizer (Perkin Elmer Co., applied Biosystems Division, Missisatiga, ON). The primers were chosen according to published sequences of the endogenous and exogenous gp85^{env} gene (Bova, C. A., et al., J. Virol., 62:75-83 (1988)) (Table 1). One set of primers, designated PUI (SEQ ID No: 15) and PU2 (SEQ ID No: 16), was a degenerate primer set designed to detect avian leukosis viral subgroups A, B, C, D, and E. Primer sets PU1/PU2 were chosen at highly conserved regions, which flanked the hypervariable region 1 for all avian leukosis/sarcoma virus subgroups in the gp85^{env} gene. A set of primers called PA1(SEQ ID No: 7) and PA2(SEQ ID No: 8) was designed in the same region as the PU1 and PU2 primers, however, the composite nucleotide sequences were specific for avian leukosis/sarcoma virus subgroup A.

| | |
|---|---|
| PA1-5′CTACAGCTGTTAGGTTCCCAGT 3′ | (SEQ ID No: 7) |
| and | |
| PA2-5′GCCTATCCGCTGTCACCACTG 3′. | (SEQ ID No: 8) |

The next five sets of primers were located in the hypervariable region 1 (hr 1) for sense and in the hypervariable region 2 (hr 2) for antisense. These primer sets were PA10 and PA20 for detection of avian leukosis/sarcoma virus subgroup A, PB1 and PB2 for detection of ALSV subgroup B, PC1 and PC2 for detection of avian sarcoma virus subgroup C, PD1 and PD2 for detection of avian sarcoma virus subgroup D, and PE1 and PE2 for detection of ALSV subgroup E.

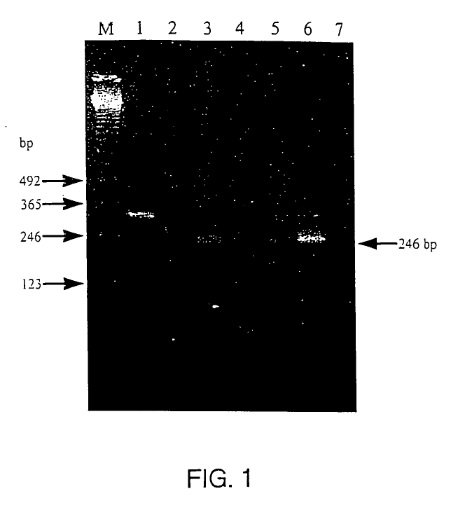

Primer set PU1 (SEQ ID No: 15) and PU2 (SEQ ID No: 16), designed to detect all avian leukosis/sarcoma virus subgroups, amplified a product of approximately 246 base pairs in 15/15 (100%) egg albumen samples from stock F chickens (Table 2, FIG. 1). The three negative controls with no RNA template and all five egg albumen specimen of non-exposed stock N chickens were negative by RT-PCR (Table 2).

Egg albumen samples of stock F and N birds were also tested using primer set PA1 (SEQ ID No: 7) and PA2 (SEQ ID No: 8) designed to recognize avian leukosis/sarcoma virus subgroup A virus. PA1 and PA2 primers amplified a fragment of 246 bp in 12/15 (80%) samples from ALSV-exposed stock F chickens. The specificity of the RT-PCR product was confirmed, as no viral amplifications were detected in the three samples containing no RNA template nor the RNA isolated from five samples of egg albumen of non-exposed stock N chickens.

The 3 samples that were positive by PU1/PU2 primer sets but negative by PA1/PA2 primer sets were also used in the RT-PCR reaction using the 5 primer sets corresponding to the hr 1 and hr 2 ALSV gp85^{env} (Table 1). No positive reactions were detected for any of the three samples.

TABLE 1

Target ALSV subgroups and oligonucleotide
primers used in RT-PCR assays

| Target | | | | | Product |
|---|---|---|---|---|---|
| Subgroup | Primer | Sequence (5′---3′)^{a} | Orientation | Position^{b} | bp |
| A | PA1 | CTACAGCTGTTAGGTTCCCAGT | Forward | gp85 of env | 229 |
| | | (SEQ ID No: 7) | | (5564-5586) | |
| | PA2 | GTCACCACTGTCGCCTATCCG | Reverse | gp85 of env | |
| | | (SEQ ID No: 8) | | (5772-5791) | |
| A, B, C, | PU1 | CTRCARCTGYTAGGYTCCCAGT | Forward | gp85 of env | 229 |
| D, E | | | | (5564-5585) | |
| | PU2 | GYCAYCACTGTCGCCTRTCCG | Reverse | gp85 of env | |
| | | | | (5772-5791) | |
| A | PA10 | GGCTTCAGGCCAAAAGGGGT | Forward | hr1 of gp85 | 232 |
| | | | | (5642-5661) | |
| | PA20 | GTGCATTGCCACAGCGGTACTG | Reverse | hr2 of gp85 | |
| | | | | (5858-5879) | |
| B | PB1 | GGCTTTACCCCATACGATAG | Forward | hr1 of gp85 | 259 |
| | | | | (5642-5661) | |
| | PB2 | ACACATCCTGACAGATGGACCA | Reverse | hr2 of gp85 | |
| | | | | (5861-5882) | |
| C | PC1 | TATTTCGCCCCAAGGGCCAC | Forward | hr1 of gp85 | 238 |
| | | | | (5642-5661) | |
| | PC2 | CCACGTCTCCACAGCGGTAAGT | Reverse | hr1 of gp85 | |
| | | | | (5858-5879) | |
| D | PD1 | GGCTTCACCCCATACGGCAG | Forward | hr1 of gp85 | 258 |
| | | | | (5642-5661) | |
| | PD2 | CCATACGTCCTCACAGATAGAATA | Reverse | hr1 of gp85 | |
| | | | | (5858-5882) | |
| E | PE1 | GGCTTCGCCCCACACTCCAA | Forward | hr1 of gp85 | 265 |
| | | | | (5642-5661) | |
| | PE2 | GCACATCTCCACAGGTGTAAAT | Reverse | hr1 of gp85 | |
| | | | | (5867-5888) | |

^{a}R = A/G; Y = T/C.
^{b}Position of primers corresponds to the numbering of the RNA genome of the Prague C strain of RSV (Schwartz et al. Cell, 32:853-869 1983).

### EXAMPLE 6

Reverse Transcription and Polymerase Chain Reaction Assay for ALSV

An RT-PCR assay was used to detect ALSV virus in egg albumen. Egg albumen samples from the stock Q, F and N chickens were subjected to RT-PCR in a masked fashion to minimize bias. To reduce the risks of contamination, RNA extraction of egg albumen was performed in a laboratory separate from the RT-PCR procedure, and RNase free reagents and pipette tips were used. RNAgents Total RNA Isolation System (Promega, USA) was used to extract total RNA from the egg albumen. The RT-PCR analysis was performed using the Access RT-PCR system (Promega, USA). Negative controls consisted of nuclease-free water instead of RNA templates and isolations of RNA from egg albumen obtained from eggs laid by chickens reared in an isolated facility free of exogenous viral exposure.

Synthesis of first-strand cDNA was performed using AMV Reverse Transcriptase (AMV RT) from Avian Myeloblastosis Virus (Promega, USA). The thermostable Tfl DNA Polymerase from Thermus flavus was used for synthesizing second strand cDNA and DNA amplification (Promega, USA). The RT-PCR assay included the following reagents in the reaction tube: nuclease-free water added to a final volume of 50 μl per reaction tube, 10 μl of AMV/Tfl 5× reaction buffer with a final concentration of 1×, 1 μl of dNTP mix (10 mM) with a final concentration of 0.2 mM, 50 picomoles of the forward and reverse primers, 2 ml of 25 mM MgSO₄ with a final concentration of 1 mM, 1 μl of AMV reverse transcriptase (5 units per ml) with a final concentration of 0.1 unit per ml, 1 μl of Tfl DNA polymerase (5 units per ml) with a final concentration of 0.1 unit per ml, and 2 μl of the sample that may contain isolated viral RNA.

The following primer sets were utilized: PU1 and PU2, PA1 and PA2, PA10 and PA20, PB1 and PB2, PC1 and PC2, PD1 and PD2, and PE1 and PE2 (Table 1). Optimal concentrations of primers was shown to be 50 picomoles per reaction for all except PA2 which was 25 picomoles/reaction. Two negative controls were included. One was a reagent control that contained nuclease-free water instead of RNA template. The other was a control using RNA from egg albumen of stock N chickens, a laboratory flock of chickens that were free of ALSV or other known poultry pathogens.

The following amplification profiles and annealing temperature were used for all primer sets, Amplification of first strand cDNA synthesis was for one cycle at 48° C. for 45 minutes, and one cycle at 94° C. for 2 minutes to inactivate the AMV reverse transcriptase and for the RNA/cDNA/primer denaturation. The amplification profile for second strand cDNA synthesis and PCR amplification was a total of 40 cycles with each step cycle at 94° C. for 30 seconds for denaturation, 60° C. for 1 minute for annealing, and 68° C. for 2 minutes for extension. A final extension cycle was also used at 68° C. for 7 minutes. Since RT-PCR reactions were performed overnight, an additional cycle at 4° C. was also added to keep the RT-PCR product at a stabilizing temperature.

Gel electrophoresis was performed on the amplified RT-PCR product using 3% Nusieve agarose (Sigma, Switzerland) with 2.5 mg/ml of ethidium bromide and 1× TAE buffer (20×) (Sigma, Switzerland) that is free of DNase, RNase, and proteinase. The gels were run at 70 V/cm for 2 hours and visualized using a UV transilluminator (Fisher Scientific, USA). The RT-PCR reaction was considered successful if the RT-PCR product provided by the kit was amplified (Promega, USA). The presence of the expected size DNA fragment was determined by comparing the amplified PCR product with the 123 base pairs DNA double-stranded ladder as a molecular size marker (Gibco, USA). From published data (Bova et al., 988), the expected size of the amplified DNA was 229 base pairs for primers PU1 and PU2, 229 base pairs for primers PA1 and PA2, 232 base pairs for primers PA10 and PA20, 259 base pairs for primers PB1 and PB2, 238 base pairs for primers PC1 and PC2, 258 base pairs for primers PD1 and PD2, and 265 base pairs for primers PE1 and PE2.

RT-PCR was performed on 15 egg albumen samples from the stock F chickens, 5 egg albumen samples from the stock N chickens, 14 samples from stock Q and the appropriate negative and positive controls. Reactions that produced the approximate expected size DNA fragment, as determined by the primer sets used, was considered positive. Samples that contained no amplification product, amplification product not of the expected size, or smears were classified as negative.

RT-PCR was first performed with primer set PU1 and PU2 to broadly screen for all avian leukosis/sarcoma virus viral subgroups (A, B, C, D, and E). Then, the same isolated RNA samples were re-screened for subgroup A avian leukosis/sarcoma virus by using primer set PA1 and PA2. Subgroup A primers were chosen since the prevalence of subgroup A avian leukosis/sarcoma virus is higher than any other avian leukosis/sarcoma virus subgroup (De Boer, G. F. et. al., Zootec Int., 10:32-35, (1981); Okazaki, W., et al., Avian Dis., 26:553-559 (1982)). Samples that were positive by PU1/PU2 but negative by PA1/PA2 primers were screened using primer sets PA10/PA20, PB1/PB2, PC1/PC2, PD1/PD2, and PE1/PE2.

Primer sets PU1 and PU2, designed to detect all avian leukosis/sarcoma virus subgroups, amplified a product of approximately 246 base pairs in 15/15 (100%) egg albumen samples from stock F chickens (Table 2, FIG. 1). The three negative controls with no RNA template and all five egg albumen specimen of non-exposed stock N chickens were negative by RT-PCR (Table 2).

Egg albumen samples of stocks Q, F and N birds were also tested using primer set PA1 and PA2 designed to recognize avian leukosis/sarcoma virus subgroup A virus (Table 2). PA1 and PA2 primers amplified a fragment of 246 bp in 12/15 (80%) samples from avian leukosis/sarcoma virus-exposed stock F chickens. Specificity of the RT-PCR product was confirmed, as no viral amplifications were detected in the three samples containing no RNA template nor the RNA isolated from 5 samples of egg albumen of non-exposed stock N chickens (Table 2). ALSV was detected in 14 out of 14 of the egg albumen samples from the stock Q chickens (Table 3, which also displays stock N data and a portion of the stock F chicken data).

The 3 samples that were positive by PU1/PU2 primer sets but negative by PA1/PA2 primer sets were also used in the RT-PCR reaction using the 5 primer sets corresponding to the hr 1 and hr 2 ALSV gp85^{env} (Table 1). No positive reactions were detected for any of the 3 samples.

The RT-PCR assay using primers PU1/PU2 designed for recognition of all avian leukosis/sarcoma virus viral subgroups detected virus in all eggs from ELISA/IFA-confirmed infected stock F chickens but not from ELISA negative, uninfected stock N chickens. Since subgroup A avian leukosis/sarcoma virus commonly infects egg-laying hens, primers PA1/PA2 designed to detect subgroup A avian leukosis/sarcoma virus was used in the second round of RT-PCR analysis to re-screen all eggs from the stock F and stock N chickens.

The RT-PCR assay using primer set PA1/PA2 detected avian leukosis/sarcoma virus in 12 of 15 eggs (80%) from stock F chickens, but did not detect virus in uninfected stock N chickens. Sequencing using primers designed for avian leukosis/sarcoma virus subgroup A verified that one of the RT-PCR amplified products was a subgroup A virus. The 202 bp product showed 84% homology to the published 205 bp sequence for RAV-1, subgroup A avian leukosis/sarcoma virus. This is the first such sequencing of a wild-type isolate of subgroup A avian leukosis/sarcoma virus by direct sequencing of the RT-PCR product, without the use of tissue culture and cloning techniques.

Primers designed in the ALSV gp85^{env} hypervariable regions (hr1 and hr2) for the five published ALSV subgroups (A-E) did not yield RT-PCR products in three samples that were positive by PU1/PU2 but negative by PA1/PA2. Since the RT-PCR assay is designed to detect ALSV in egg albumen, the potential wild-type virus in these 3 samples may not exhibit the same nucleotide sequences in the hypervariable regions as the published ALSV sequences which limits the specificity of hr 1 and hr 2 primers.

TABLE 2
RT-PCR analysis using primer sets PU1 and PU2 for detecting ALSV
viral subgroups and PA1 and PA2 for detecting ALSV subgroup A of
egg albumen from ALSV-positive stock F and ALSV-negative stock N
White Leghorn chickens.

RT-PCR results (samples positive/tested)

| | PU1 and PU2 primers | PA1 and PA2 |
|---|---|---|
| Chicken | (subgroup A, B, C, D, E) | (subgroup A) |
| 1. F | +(3/3) | +(3/3) |
| 2. F | +(3/3) | +(3/3) |
| 3. F | +(3/3) | +(3/3) |
| 4. F | +(3/3) | +(2/3) |
| 5. F | +(3/3) | +(1/3) |
| 6. N | −(0/1) | −(0/1) |
| 7. N | −(0/1) | −(0/1) |
| 8. N | −(0/1) | −(0/1) |
| 9. N | −(0/1) | −(0/1) |
| 10. N | −(0/1) | −(0/1) |

TABLE 3
Detection of ALSV by ELISA, IFA, and RT-PCR.

| Chicken^{a} | | IFA^{c} | | | RT-PCR^{d} |
|---|---|---|---|---|---|
| Number/ | ELISA^{b} | Egg | Feather | | Egg |
| strain | Egg albumen | albumen | pulp | Blood | albumen |
| 7Q | + | − | − | − | +(3/3) |
| 8Q | + | − | − | − | +(3/3) |
| 9Q | + | − | − | − | +(3/3) |
| 10Q | + | − | − | − | +(2/2) |
| 11Q | − | − | − | − | +(3/3) |
| 2F | + | + | + | + | +(3/3) |
| 6F | − | − | − | − | +(2/3) |
| 1N | − | NT | NT | NT | −(0/1) |
| 2N | − | NT | NT | NT | −(0/1) |
| 3N | − | NT | NT | NT | −(0/1) |
| 4N | − | NT | NT | NT | −(0/1) |
| 5N | − | NT | NT | NT | −(0/1) |

^{a}Q and F: strains of chickens naturally exposed to ALSV; laboratory reared N flock of chickens were free of ALSV.
^{b}+: presence of ALSV gsa; −: absence of ALSV gsa as detected by ELISA.
^{c}IFA tests were run in duplicate which were 100% in agreement for both positive and negative results; NT: not tested, + denotes IFA detection of gsa from replicating ALSV in C/E cells inoculated with egg albumen, feather pulp, or blood; − denotes absence of ALSV gsa in C/E cells as detected by IFA.
^{d}primer set PA1 and PA2 to detect subgroup A ALSV, was used, in the RT-PCR assay; +: RT-PCR positive reaction for one or more eggs tested per chicken, with numbers in parentheses showing number of positive samples over total number tested.

### EXAMPLE 7

Survey of Eggs Sold by Retail Stores in New Orleans, La., USA.

A telephone survey of the retail stores in the New Orleans Metropolitan Area was conducted between December 1996 and February 1997. The sampling frame was grocery stores listed in the New Orleans Yellow Pages for 1995/1996, and the respondents were individuals who either owned, managed, or ordered eggs for the store. A total of 417 grocery stores in the New Orleans area were listed in the Yellow Pages telephone directory (1995/1996). Of these 64 (15%) stores were no longer in existence. Of the remaining 353 grocery stores, 74 retail stores did not sell eggs. Of the 279 stores which did sell eggs, telephone surveys were completed for 275 retail stores (a participation rate of 99%). A total of 275 grocery stores sold 174,871 dozens of eggs per week or 8.4 million eggs per month in New Orleans, La. Based on an expected 20% ALSV prevalence, the equation N=Z²_{0.975} (p)(1−p)/d² (Rosner B., Fundamentals of Biostatistics, 4th Edition, (Duxbury Press, Wadsworth Publishing Company, U.S.A, 1995) was used to determine the sample size of 240 chicken eggs from New Orleans retail store.

### EXAMPLE 8

Testing Commercial Chicken Eggs for ALSV

RT-PCR was also used to determine the prevalence of ALSV in chicken eggs being sold by retail stores in New Orleans, La. Based on an expected ALSV prevalence of 20% (Okazaki W., et al., Avian Diseases, 26:553-559 (1982); Rosner B., Fundamentals of Biostatistics. 4th Edition, (Duxbury Press, Wadsworth Publishing Company, U.S.A, (1995)), one dozen eggs were tested from each of 20 randomly chosen stores (Table 4). One or more infected eggs were detected in 80% of the egg cartons sampled. Each carton of 12 eggs contained an average of 1.7 infected eggs. Overall, 14.2% of 240 eggs were infected. Based on this survey, it is estimated that at least 1.2 of 8.4 million eggs being sold per month in New Orleans are infected with avian leukosis/sarcoma virus. This finding was disturbing, as the poultry industry maintains an intense avian leukosis/sarcoma virus eradication program (e.g. Spencer J. L., et al., Avian Diseases 28:358-373 (1984)). This is the first time such an estimate of the molecular prevalence of avian leukosis/sarcoma virus infection in commercial eggs was determined from a statistically based sample.

TABLE 4
RT-PCR results of eggs obtained from retail stores using
oligonucleotide primers PA1/PA2

| | Storage | | | |
|---|---|---|---|---|
| Store | Temperature | Number of | Number of eggs | Percent |
| Number | of eggs (° F.)^{a} | eggs tested | positive for ALSV | positive |
| 2 | 39.2 | 12 | 3 | 25.0 |
| 20 | 44.6 | 12 | 3 | 25.0 |
| 22 | 39.2 | 12 | 1 | 8.3 |
| 29 | 42.8 | 12 | 2 | 16.7 |
| 40 | 42.8 | 12 | 1 | 8.3 |
| 49 | 41.0 | 12 | 2 | 16.7 |
| 56 | 46.4 | 12 | 1 | 8.3 |
| 63 | 46.4 | 12 | 1 | 8.3 |
| 64 | 33.8 | 12 | 2 | 16.7 |
| 65 | 39.2 | 12 | 1 | 8.3 |
| 67 | 39.2 | 12 | 0 | 0.0 |
| 68 | 44.6 | 12 | 0 | 0.0 |
| 73 | 39.2 | 12 | 3 | 25.0 |
| 79 | 35.6 | 12 | 3 | 25.0 |
| 88 | 46.4 | 12 | 0 | 0.0 |
| 89 | 39.2 | 12 | 2 | 16.7 |
| 202 | 46.4 | 12 | 2 | 16.7 |
| 207 | 59.0 | 12 | 3 | 25.0 |
| 246 | 39.2 | 12 | 3 | 25.0 |
| 265 | 39.2 | 12 | 1 | 8.3 |

^{a}Storage temperature of commercial chicken eggs were determined by on-site investigations of 20 randomly chosen grocery stores.

### EXAMPLE 9

Sequencing RT-PCR Products

For purification prior to sequencing, RT-PCR generated products electrophoresed as above were electroeluted from the gel using an electroeluter (Model UEA, IBI). Sequencing was performed by Commonwealth Biotechnologies, Inc., (Virginia, USA) using an ABI Prism DNA sequencer Model 377 (Perkin Elmer, USA) and the ABI Prism Dye Terminator Cycle Sequencing Ready Reaction Kit (Perkin Elmer, USA). Both sense (PA1) (SEQ ID No: 7) and antisense (PA2) (SEQ ID No: 8) primers for subgroup A ALSV were used in the sequencing reactions. Nucleotide sequences generated from the sequenced samples were compared to the published viral subgroup A, B, C, D, and E sequences (Bova, C. A., et al., J. Virol., 62:75-83 (1988)) using the computer program Sequencher (AGTC Inc., USA). In addition, multiple sequence alignments were produced using MacVector Clustral W (1.4) (Oxford Molecular, California, USA).

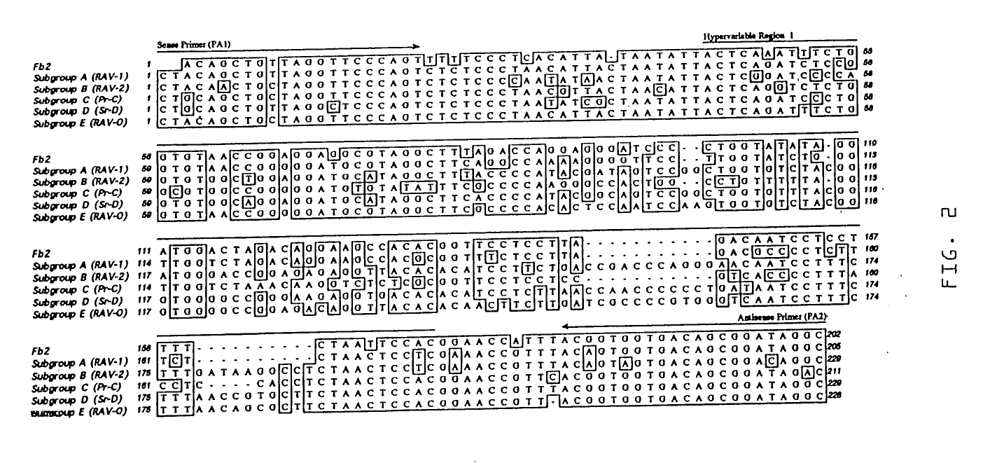

Direct sequencing using primer set PA1 and PA2 was performed on sample Fb2, an RT-PCR product amplified from the egg albumen of the number 2 F chicken that tested positive for ALSV by ELISA and IFA. Sample Fb2 was positive by RT-PCR analyses using both primer sets PU1/PU2 and PA1/PA2. Direct sequencing of sample Fb2 was performed using sense primer PA1 and antisense primer PA2, which resulted in 100% similarity in both the sense and antisense nucleotide sequences. Sequence analysis of sample Fb2 provided a nucleotide sequence of 202 bp. The sequence data for sample Fb2 (SEQ ID No: 6) showed an 84% homology to RAV-1, subgroup A ALSV (SEQ ID No: 1), 57% homology to RAV-2, subgroup B ALSV (SEQ ID No: 2), 71% homology to PR-C, subgroup C ALSV (SEQ ID No: 3), 59% homology to Sr-D, subgroup D ALSV (SEQ ID No: 4), and 61% homology to RAV-0, subgroup E ALSV (SEQ ID No: 5) (FIG. 2). In addition, direct sequencing of the three samples that were negative by PA1/PA2 primers did not amplify any RT-PCR products, indicating that the samples did not contain ALSV subgroup A virus.

Randomly selected egg albumen samples positive for ALSV by RT-PCR were subjected to direct nucleotide sequencing (FIG. 1, Table 5). Nucleotide sequences of sample 6F (SEQ ID No: 10) from stock F chickens, 7Q (SEQ ID No: 11) and 10Q (SEQ ID No: 12) from stock Q chickens, and sample 207 (SEQ ID No: 13) from New Orleans retail stores showed a ≧95% homology to each other and had a ≧95% homology with RAV-O, the prototype for subgroup E ALSV (SEQ ID No: 5). Both sample 2F (SEQ ID No: 9) from stock F chickens and sample 65 (SEQ ID No: 14) from New Orleans retail stores shared 95% homology to each other and had an 84% homology to RAV-1, the prototype for subgroup A ALSV (SEQ ID No: 1).

TABLE 5
ALSV viral subgroup classification of six sequenced RT-PCR
products from egg albumen

| | | | | | Viral sub- |
|---|---|---|---|---|---|
| | | | | | group and |
| | | | | | sequence |
| Origin of egg | Egg | Analysis for ALSV | | | homology |
| albumen | albumen | ELISA^{a} | IFA^{b} | RT-PCR^{c} | (%)^{d} |
| Stock F chickens | 2F | + | + | + | A (84%) |
| Stock F chickens | 6F | − | − | + | E (95%) |
| Stock Q chickens | 7Q | + | − | + | E (95%) |
| Stock Q chickens | 10Q | + | − | + | E (99%) |
| Commercial egg | 207 | NT | NT | + | E (96%) |
| Commercial egg | 65 | NT | NT | + | A (84%) |

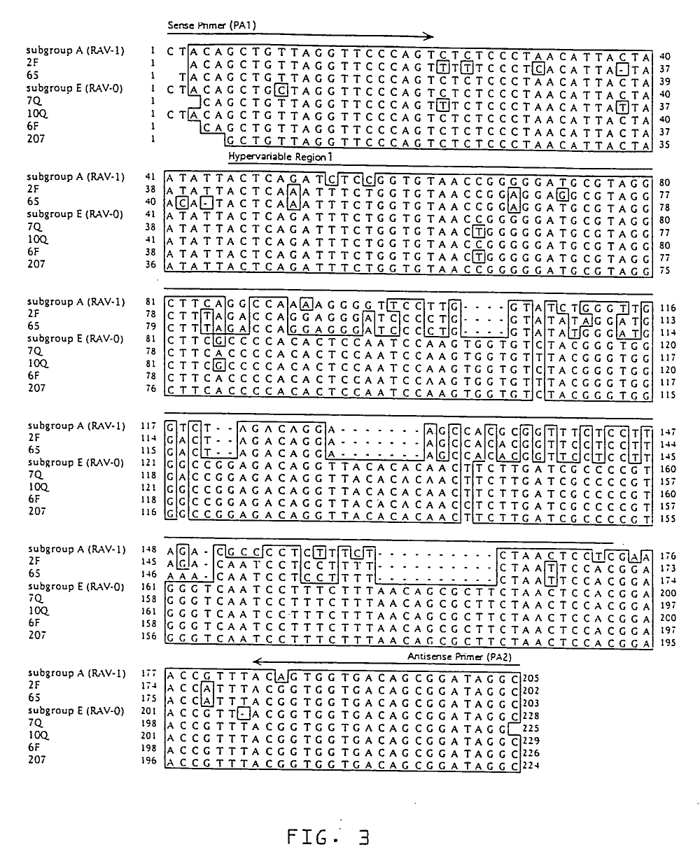

^{a}+: presence of ALSV gsa, while − denotes absence of ALSV gsa as detected by ELISA; NT: not tested.
^{b}+: gsa of replicating ALSV in C/E cells inoculated with egg albumen, while − absence of ALSV gsa in C/E cells as detected by IFA.
^{c}RT-PCR analysis was performed using primer set PA1 and PA2 on isolated RNA from albumen of chicken eggs.
^{d}Subgroup classification based on percent sequence homology compared with the published viral sequences. See FIG. 3.

### EXAMPLE 10

Presence of Endogenous ALSV in Unfertilized Chicken Eggs

It was expected that the RT-PCR would detect subgroup A ALSV because the prevalence of subgroup A ALSV infections in chickens is higher than subgroup B ALSV. R while reports of infections of subgroup C ALSV and subgroup D ALSV in chickens have been more common in countries outside the U.S., such as Finland (Calnek, et al., Diseases of Poultry. 9th Ed, I.S.U. Press, Ames, Iowa, (1991). However, subgroup E avian leukosis virus was not expected in commercial chicken eggs since shedding of endogenous ALSV from mother hen to egg rarely occurs, as indicated in experiments with K28 chickens (Robinson, et al., Science 225:417-419 (1984)).

As discussed supra, nucleotide sequence analysis performed on randomly selected RT-PCR products of albumen from eggs obtained from commercial chickens and from retail stores showed samples were positive for endogenous ALSV. This finding of endogenous subgroup E ALSV in unfertilized chicken eggs at such a high frequency was an unexpected one. It appears as though the congenital transmission of ALSV is common and should be considered in the implementation of ALSV eradication programs by the poultry industry. These findings suggest that endogenous ALSV shedding into the albumen of unfertilized eggs may be much more common than expected and that the endogenous virus is being transmitted congenitally. Thus, at least some types of endogenous retroviruses can be transmitted in a non-Mendelian fashion.

Any publications mentioned in this specification are indicative of the levels of those skilled in the art to which the invention pertains. Further, these publications are incorporated by reference herein to the same extent as if each individual publication was specifically indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, molecules, and specific compounds described herein are presently representative of preferred embodiments, are exemplary, and are not limitations on the scope of the invention. Changes therein and other uses will occur to those skilled in the art which are encompassed within the spirit of the invention as defined by the scope of the claims.

1. A method of detecting avian leukosis/sarcoma viruses at the nucleic acid level in an avian sample, comprising the steps of:
isolating viral RNA from said avian sample; and
performing RT-PCR.

2. The method of claim 1, wherein said avian sample is selected from the group consisting of unfertilized chicken egg albumen, fertilized chicken egg albumen, unfertilized egg albumen from an animal of the class A ves and fertilized egg albumen from an animal of the class Aves.

3. The method of claim 1, wherein said avian sample is blood from an animal of the class Aves.

4. The method of claim 1, wherein said avian sample is feather pulp from an animal of the class Aves.

5. The method of claim 1, wherein said avian sample is a cell, tissue or body fluid from an animal of the class Aves.

6. A method of determining avian leukosis/sarcoma virus subgroup specificity at the nucleic acid level, including distinguishing between exogenous and endogenous retroviruses, comprising the steps of:
isolating viral RNA a specimen from an avian sample;
performing RT-PCR; and
sequencing the amplified RT-PCR product.

7. The method of claim 6, wherein said avian sample is selected from the group consisting of unfertilized chicken egg albumen, fertilized chicken egg albumen, unfertilized egg albumen from an animal of the class Aves and fertilized egg albumen from an animal of the class Aves.

8. The method of claim 6, wherein said avian sample is blood from an animal of the class Aves.

9. The method of claim 6, wherein said avian sample is feather pulp from an animal of the class Aves.

10. The method of claim 6, wherein said avian sample any cell or tissue or body fluid from an animal of the class Aves.

11. An oligonucleotide specific for the detection of viral subgroup A of avian leukosis/sarcoma virus at the nucleic acid level, said oligonucleotide having a sequence at least 95% identical to a sequence selected from the group consisting of:
(a) SEQ ID No: 7 and SEQ ID No: 8;
(b) a nucleotide sequence encoding the gp^{env} 85 protein; and
(c) an oligonucleotide which hybridizes under stringent hybridization conditions to a oligonucleotide defined by (a) or (b).

12. An oligonucleotide specific for the detection of viral subgroups A-E of avian leukosis/sarcoma virus at the nucleic acid level, said oligonucleotide having a sequence at least 95% identical to a sequence selected from the group consisting of:
a) SEQ ID No: 15 and SEQ ID No: 16;
b) a nucleotide sequence encoding the gp^{env} 85 protein; and
c) an oligonucleotide which hybridizes under stringent hybridization conditions to an oligonucleotide defined by (a) or (b).

13. A method of detecting avian leukosis/sarcoma viruses at the nucleic acid level in a poultry sample, comprising the steps of:
isolating viral RNA from said poultry sample; and
performing RT-PCR using an oligonucleotide of claim 11.

14. A method of detecting avian leukemia viruses at the nucleic acid level in a poultry sample, comprising the steps of:
isolating viral RNA from said poultry sample; and
performing RT-PCR using an oligonucleotide of claim 12.

15. A method of determining avian leukemia virus subgroup specificity at the nucleic acid level and distinguishing between exogenous and endogenous retroviruses, comprising the steps of:
obtaining a specimen from a poultry sample;
isolating viral RNA from said sample;
performing RT-PCR using an oligonucleotide of claim 11; and
sequencing the amplified RT-PCR product.

16. A method of determining avian leukosis/sarcoma virus subgroup specificity at the nucleic acid level and distinguishing between exogenous and endogenous retroviruses, comprising the steps of:
obtaining a specimen from a poultry sample;
isolating viral RNA from said sample;
performing RT-PCR using the primers of claim 12; and
sequencing the amplified RT-PCR product.
